(19) United States
(12) Patent Application Publication
(10) Pub. No.: US 2022/0282237 A1
(43) Pub. Date: Sep. 8, 2022

(54) NOVEL FUCOSE ISOMERASE AND FUCOSE PRODUCTION METHOD USING SAME

(71) Applicant: Korea University Research and Business Foundation, Seoul (KR)

(72) Inventors: Kyoung Heon KIM, Seoul (KR); Ki Hyun NAM, Seoul (KR); Do Hyoung KIM, Yongin-si (KR); In Jeong KIM, Seoul (KR)

(73) Assignee: Korea University Research and Business Foundation, Seoul (KR)

(21) Appl. No.: 17/637,277

(22) PCT Filed: Aug. 25, 2020
(86) PCT No.: PCT/KR2020/011336

§ 371 Date: Feb. 22, 2022

(30) Foreign Application Priority Data
Aug. 29, 2019 (KR) 10-2019-0106598

(51) Int. Cl. C12N 9/90; C12P 19/24

(52) U.S. Cl. CPC C12N 9/90; C12Y 503/01025; C12P 19/24

(57) ABSTRACT

The present invention relates to a novel fucose isomerase and a fucose production method using same. More specifically, in a reversible reaction between L-fucose and L-fuculose, an L-fucose isomerase, derived from a Raoultella sp. KDH 14 strain isolated from abalone intestines, favors the reaction progressing from L-fuculose to L-fucose, and thus the present invention provides the effect of applying same to the production of L-fucose.

### BACKGROUND

### 1. Field of the Invention

The present invention relates to a fucose isomerase which reversibly catalyzes an isomerization reaction between L-fucose and L-fuculose and is derived from a novel Raoultella sp. KDH 14 strain isolated from abalone intestines, and a fucose production method using same.

### 2. Discussion of Related Art

L-fucose (6-deoxy-L-galactose) is a rare sugar that occurs in a variety of organisms, from bacteria to humans. For example, L-fucose is found in the form of a human milk oligosaccharide (HMO) or a sugar protein, and is also present as a constituent of microbial exopolysaccharides (EPSs) and seaweed. Due to its various physiologically active properties, L-fucose may be used as an anti-inflammatory, anti-tumor and immune-enhancing drug in the pharmaceutical field, as a whitening, skin moisturizing and anti-aging agent in the cosmetic industry, or as a nutrient. Since L-fucose has various physiological activities, L-fucose can be applied to various fields such as pharmaceuticals, medicine, food, and cosmetics.

For industrial applications, L-fucose can be produced by three major methods (hydrolysis of polysaccharides, and chemical and enzymatic synthesis). First, the method of hydrolyzing polysaccharides is a method of obtaining fucose by treating exopolysaccharides of seaweed or microorganisms containing fucose with an acid or an enzyme. Second, the chemical synthesis is a method of synthesizing L-fucose from inexpensive sugars such as D-galactose or D-mannose using chemicals. The above two methods have disadvantages in that the yield is low, by-products are produced, and a lot of labor is required. That is, economic utility is low. In contrast, the enzymatic synthesis method can specifically produce L-fucose, is environmentally friendly, and is considered to be cost-efficient. There are two major known methods of synthesizing L-fucose based on the enzymatic method, and L-fuculose is involved as an intermediate in both the reactions. One is a method based on an aldol reaction during a metabolic process of L-fucose, and after an aldol reaction between lactaldehyde and dihydroxyacetone phosphate (DHAP) and then dephosphatation by acid phosphatase, L-fuculose is produced. The second is an enzymatic-chemical method, and first, L-fucitol is synthesized by a chemical method using D-galactose as a starting material, and then is converted into L-fuculose by a dehydrogenase. Both methods require an L-fucose isomerase (EC 5.3.1.25) to convert an intermediate L-fuculose into L-fucose. L-FucI is a type of ketol isomerase that catalyzes an interconversion between L-fucose and L-fuculose. The reaction between L-fucose and L-fuculose is reversible, and there is a limit to an increase in the production yield of L-fucose by reaching an equilibrium state. Therefore, the use of L-FucI, which favors the reverse reaction, that is, the reaction from L-fuculose to L-fucose, is advantageous for the enzymatic production of L-fucose. Although the enzymatic method is attractive as described above, there are only two cases of studying the reversible reaction between L-fucose and L-fuculose to date. Although the study cases more favorably catalyzed the reverse reaction, they did not study L-fuculose as a substrate. In fact, in order to enhance the applicability of an isomerase for the synthesis of L-fucose, studies using L-fuculose as a substrate are required but such studies have not been reported. Therefore, it is important to discover a new isomerase for which the reverse reaction is dominant, examine the reversible reaction of the isomerase, and investigate biochemical properties using L-fuculose as a substrate.

### SUMMARY OF THE INVENTION

An object of the present invention is to provide a new microorganism isolated from abalone intestines using fucoidan as a carbon source.

Another object of the present invention is to provide a novel L-fucose isomerase which is isolated from the microorganism and favors a conversion reaction from L-fuculose to L-fucose in a reversible reaction between L-fucose and L-fuculose, and a preparation method thereof.

Still another object of the present invention is to provide a method for producing L-fucose using the novel microorganism and an L-fucose isomerase derived therefrom.

In order to achieve the objects, the present invention provides an L-fucose isomerase which includes the amino acid sequence of SEQ ID NO: 1 and favors a conversion reaction from L-fuculose to L-fucose in a reversible reaction between L-fuculose and L-fucose.

The present invention also provides a nucleic acid molecule encoding the L-fucose isomerase.

The present invention also provides a recombinant vector including the nucleic acid molecule.

The present invention also provides a host cell transformed with the recombinant vector.

The present invention also provides a method for preparing an L-fucose isomerase, the method including: expressing an L-fucose isomerase by culturing the host cell; and obtaining the expressed L-fucose isomerase.

The present invention also provides a composition for producing L-fucose including the L-fucose isomerase; and one or more substrates selected from the group consisting of L-fuculose and D-ribulose.

The present invention also provides a method for producing L-fucose, the method including: reacting the L-fucose isomerase with one or more substrates selected from the group consisting of L-fuculose and D-ribulose.

The L-fucose isomerase derived from a Raoultella sp. KDH 14 strain isolated from abalone intestines according to the present invention favors the reaction progressing from L-fuculose to L-fucose in a reversible reaction between L-fucose and L-fuculose, and thus can be applied to the production of L-fucose.

### BRIEF DESCRIPTION OF THE DRAWINGS

FIG. 1 shows a 16S rRNA-based phylogenetic tree of the Raoultella sp. KDH 14 strain of the present invention.

FIG. 2 shows a RdFucI gene sequence derived from the Raoultella sp. KDH 14 strain of the present invention.

FIG. 3 shows the gel filtration chromatography results of RdFucI derived from the Raoultella sp. KDH 14 strain of the present invention and the SDS-PAGE results (insert) purified by affinity chromatography using a His-Trap column.

FIG. 4a shows the enzymatic conversion from L-fucose to L-fuculose (forward reaction) by RdFucI derived from the Raoultella sp. KDH 14 strain of the present invention.

FIG. 4b shows the enzymatic conversion from L-fuculose to L-fucose (reverse reaction) by RdFucI derived from Raoultella sp. KDH 14 strain of the present invention.

FIG. 5a shows the effect of temperature on fuculose of RdFucI derived from the Raoultella sp. KDH 14 strain of the present invention.

FIG. 5b shows the effect of pH on fuculose of derived from the Raoultella sp. KDH 14 strain of the present invention.

FIG. 6 shows the substrate specificity of RdFucI derived from the Raoultella sp. KDH 14 strain of the present invention.

FIGS. 7a and 7b show the entire structure and oligomeric form of RdFucI derived from the Raoultella sp. KDH 14 strain of the present invention, and

FIG. 7a is a cartoon representation of a RdFucI monomer, and the RdFucI monomer consists of N1-(yellow), N2-(pink) and C-(green) domains.

FIG. 7b is a surface representation of a RdFucI hexamer, and subunits A, B, C, D, E and F are indicated by yellow, pink, turquoise, purple, green and orange, respectively. A metal binding site on a substrate binding pocket site is indicated by a red dot.

FIGS. 8a to 8f show the binding recognition and active sites of RdFucI derived from the Raoultella sp. KDH 14 strain of the present invention, and

In FIG. 8a, the substrate binding pocket is formed by assembly with Subunits A and C.

FIG. 8b shows the electrostatic surface of the substrate binding pocket.

FIG. 8c suggests a B-factor on the substrate binding surface.

FIG. 8d is a cross-sectional surface view of the substrate binding pocket. A 2Fo-F electron density map for the metal binding site of RdFucI (gray mesh, contoured at 1.0σ) is immersed in a solution including 10 mM Mn²⁺.

FIG. 8f is a geometric analysis result of the Mn²⁺ binding site of RdFucI.

FIGS. 9a to 9d show the structural differences between RdFucI derived from the Raoultella sp. KDH14 strain of the present invention and the existing FucI.

FIGS. 9a to 9d show active sites of EdFucI (PDB code: 1FUI), ApFucI (3A9R) and SpFucI (4C20) and RdFucI (FIG. 9a) and the superimposition of the substrate binding surfaces (FIG. 9b). An enlarged view of the large morphological differences of the α8-α9 loop from L-FucI was inserted (left).

FIG. 9c is a partial sequence alignment of the α8-α9 loop for RdFucI, EcFucI, ApFucI and SpFucI.

FIG. 9d is an electrostatic surface representation of RdFucI, EcFucI, ApFucI and SpFucI. Deep substrate binding pockets and α8-α9 loop regions are indicated by orange and black dots, respectively. The metal binding sites are indicated by yellow asterisks.

### DETAILED DESCRIPTION OF EXEMPLARY EMBODIMENTS

Hereinafter, the configuration of the present invention will be described in detail.

The present invention relates to an L-fucose isomerase which includes the amino acid sequence of SEQ ID NO: 1 and favors a conversion reaction from L-fuculose to L-fucose in a reversible reaction between L-fucose and L-fuculose.

The present inventors isolated a new microbial species belonging to the genus Raoultella growing on fucoidan, which is a constituent of seaweed, as the only carbon source from abalone intestines. The isolated strain was identified as a novel species of the genus Raoultella by a 165 rRNA sequencing-based phylogenetic tree, and was named Raoultella sp. KDH 14. To find an isomerase of fucose, a full-length genome sequence was confirmed by performing a genome sequence analysis, and a fucose isomerase was identified by identifying BLAST-based sequence similarity (RdFucI). Furthermore, the ratio of a mixture in the equilibrium state was examined by studying a reversible reaction between L-fucose and L-fuculose of L-FucI derived from a Raoultella sp. KDH 14 strain, and various biochemical properties (temperature, pH and metal ion effects) were investigated using L-fuculose as a substrate. Further, the mechanism of action was understood at the molecular level through structural analysis and the difference from the existing L-FucI was identified.

As a result, RdFucI includes the amino acid sequence of SEQ ID NO: 1 and is encoded by the base sequence of SEQ ID NO: 2, and showed higher activity on L-fuculose than on L-fucose, and the reaction rate was about 5-fold faster in the reaction from L-fuculose to L-fucose. In the equilibrium state, L-fucose and L-fuculose were present at a ratio of about 9:1. Such results show that a reaction catalyzed by RdFucI much more favors the production of L-fucose. Such characteristics are advantageous for industrial production of L-fucose with L-FucI.

The L-fucose isomerase of the present invention favors the reaction progressing from L-fuculose to L-fucose in a reversible reaction between L-fucose and L-fuculose, and thus exhibits a proportion of L-fucose of 90% or more in the equilibrium state even when either L-fucose or L-fuculose is used as a substrate.

In addition, when L-fuculose is used as a substrate, a relative maximum enzyme activity of 80% or more is exhibited at 30 to 50° C. However, when the temperature is out of the above temperature range, the enzyme activity drops to less than 50%. Furthermore, a relative maximum activity of 70% or more is exhibited at a pH of 6 to 11 in a sodium acetate buffer, a sodium phosphate buffer and a glycine-NaOH buffer, whereas in the case of a Tris-HCl buffer, the activity was low under the above pH conditions, meaning that Tris may act as an inhibitor of the enzyme.

Further, in general, sugar isomerases require a metal ion as a cofactor, and the L-fucose isomerase of the present invention exhibits high relative activity during a reaction under one or more metal ions selected from the group consisting of Mn²⁺, Mg²⁺, Co²⁺, Cd²⁺ and Zn²⁺. In particular, the activity of the enzyme is increased about 7-fold higher in the presence of Mn²⁺. In addition, the activity of the L-fucose isomerase of the present invention may be inhibited by one or more metal ions selected from the group consisting of Fe²⁺, Ca²⁺ and Cu²⁺ (see Table 1).

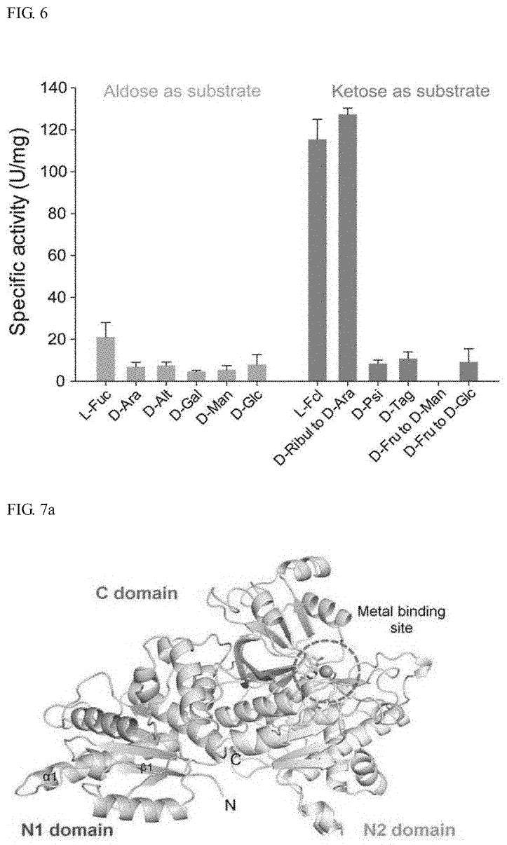

Further, in general, sugar isomerases exhibit various substrate reactivities, and the L-fucose isomerase of the present invention exhibits high activity for L-fuculose and D-ribulose, and thus shows a much more dominant reaction on a ketose substrate than on an aldose substrate(see FIG. 6).

Meanwhile, it was confirmed through crystal structure analysis that the L-fucose isomerase of the present invention has a loop conformation near an active site, which is different from the L-FucIs revealed in the related art, and it is specifically described as follows.

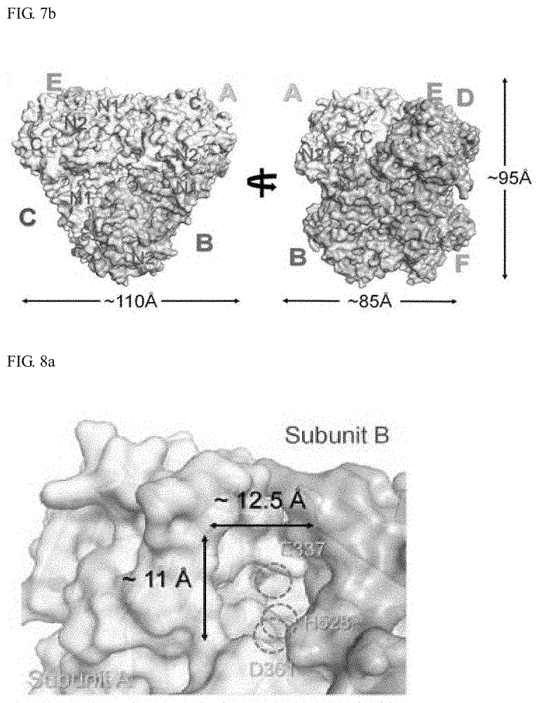

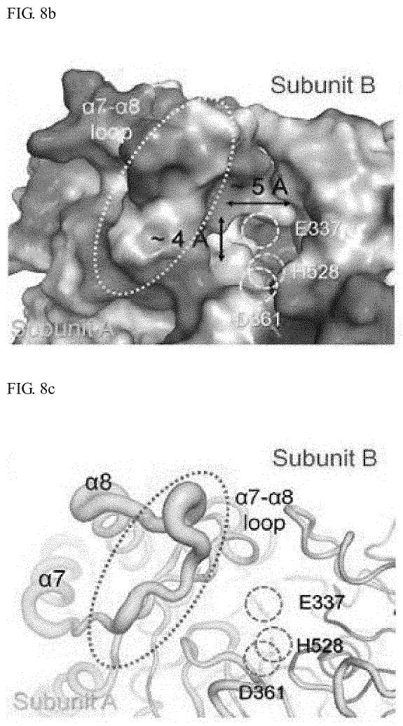

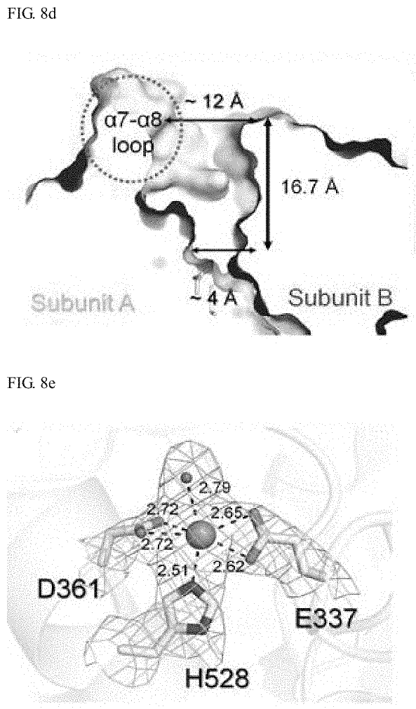

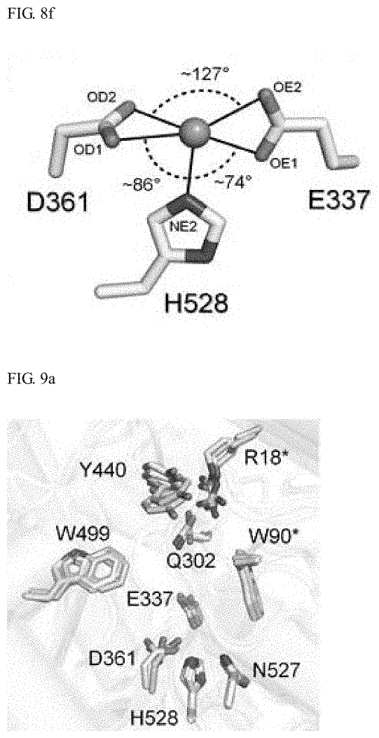

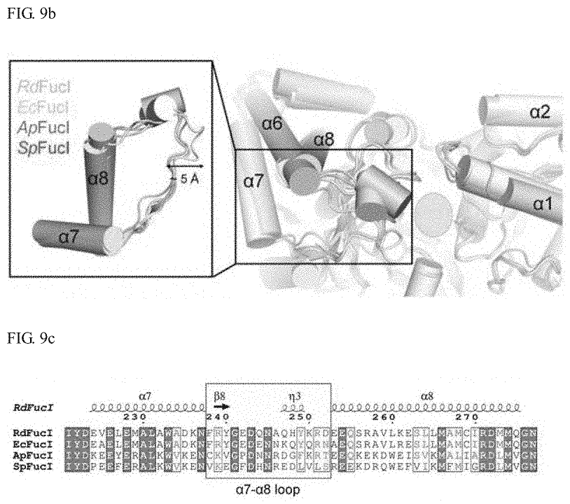

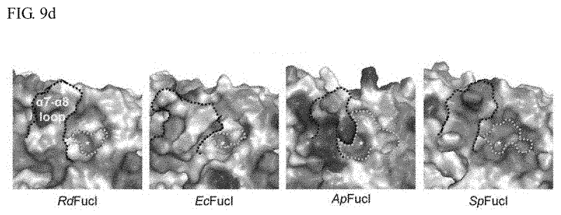

Monomeric Rd FucI includes 19 α-helices and 23 α-strands, including N1, N2 and C domains (FIG. 7A). The N1 domain (Ser5-Met172) adopts an α/β-fold and is involved in substrate recognition of the hexamer formation of RdFucI. The N2 domain (Lys173-Leu352) and the C domain (Thr353-Arg591) include metal binding residues involved in catalytic activity (FIG. 7A). The RdFucI subunit has D3h virtual symmetry with a dimer of a trimer. The substrate binding pocket is formed by the N2 and C domains of Subunit A and the N1 domain of Subunit B (FIGS. 8A to 8C), and has a total of 6 substrate binding sites in homohexameric RdFucI. An entrance of a substrate binding pocket to which a substrate approaches is about 11×12.5 Å (FIG. 8A). A substrate binding pocket, in which a metal binding site is formed, has a negatively-charged surface of about 4×5 Å (FIG. 8B). The distance between the metal binding site and the substrate binding pocket is about ˜16.7 Å (FIG. 8D), meaning that the active center is located deep in the pocket. This shows that both the open form and ring form of the substrate can access the active site center, but this also shows that bulk saccharides cannot access an active site present inside the substrate binding pocket. The superimposition of the substrate binding pockets shows that substrate recognition residues (Arg16; W88, Gln300, Tyr437, Trp496 and Asn524) exhibit minor three-dimensional structures, whereas metal binding residues Glu337, Asp361, and His528 (numbered in RdFucI) are positionally identical to other proteins. In contrast, the α7-α8 loop of each L-FucI placed on the surface of the substrate binding pocket has a different conformation. Although the sequence alignment of L-FucI exhibits high similarity, the sequence of the α7-α8 loop of each L-FucI is very diverse (FIG. 9B). Each L-FucI forms a unique substrate binding pocket because the α7-α8 loop is involved in the structure formation of the substrate binding pocket (FIG. 9C). Although L-FucI generally has a negatively charged surface around the metal binding site, the surface of the substrate binding pocket exhibits a different charge state (FIG. 9C). As a result, a difference in the α7-α8 loop structure may cause a difference in the substrate specificity of L-FucI.

Further, the L-fucose isomerase of the present invention may be transcribed and translated through not only a region before and after a coding region of the enzyme, but also a DNA segment associated with production of a polypeptide including an intervening sequence between individual coding segments, that is, a coding gene. For example, the L-fucose isomerase may be transcribed and translated from the sequence set forth in SEQ ID NO: 2, but is not particularly limited thereto. Further, a protein that favors a conversion reaction from L-fuculose to L-fucose in a reversible reaction between L-fucose and L-fuculose as a variant protein with one or more of substitution, deletion, transposition, addition, and the like of the enzyme is also included in the scope of the enzyme of the present invention, and preferably, includes an amino acid sequence having a sequence identity of 80% or more, 85% or more, 90% or more, 93% or more, 94% or more, 95% or more, 96% or more, 97% or more, 98% or more, and 99% or more with the amino acid sequence set forth in SEQ ID NO: 1.

In the present specification, “protein” and “polypeptide” are used interchangeably in the present application.

In the present invention, the fact that a polypeptide has a sequence identity of a specific percentage (for example, 80%, 85%, 90%, 95%, or 99%) with another sequence means that when the two sequences are aligned, there is a specific percentage of identical amino acid residues at the time of comparing the sequences. The alignment and percentage homology or identity may be determined using those described in any suitable software program publicly known in the art, for example, a document [CURRENT PROTOCOLS IN MOLECULAR BIOLOGY (F. M. Ausubel et al., (eds) 1987 Supplement 30 section 7.7.18)]. Examples of a preferred program include a GCG Pileup program, FASTA (Pearson et al., 1988 Pr° C. Natl Acad. Sci USA 85:2444-2448), and BLAST (BLAST Manual, Altschul et al., Natl. Cent. Biotechnol. Inf., Natl Lib. Med. (NCIB NLM NIH), Bethesda, Md., and Altschul et al., 1997 NAR25:3389-3402). Another preferred alignment program is ALIGN Plus (Scientific and Educational Software, PA), and preferably, is an alignment program which uses base parameters. Another available sequence software program is the TFASTA Data Searching Program available in the Sequence Software Package Version 6.0 (Genetics Computer Group, University of Wisconsin, Madison, Wis.).

The L-fucose isomerase may be isolated and purified from supernatants of a culture of the new strain Raoultella sp. KDH 14 of the present invention using fucoidan as a carbon source, and may be produced and isolated by strains other than Raoultella sp. KDH 14 using genetic engineering recombination technology or an artificial chemical synthesis method.

When recombinant technology is used, factors used in order to facilitate the expression of a typical recombinant protein, such as an antibiotic resistance gene, and a reporter protein or a peptide usable for affinity column chromatography may be used, and these techniques fall within the scope that can be easily carried out by a person skilled in the art to which the present invention pertains. For example, the L-fucose isomerase may be obtained from a host cell transformed with a recombinant vector including a gene encoding the L-fucose isomerase, that is, the base sequence set forth in SEQ ID NO: 2 or a culture thereof. Escherichia coli is used as the host cell, but host cell is not limited thereto.

The L-fucose isomerase may use L-fuculose, D-ribulose, and the like as a substrate.

The present invention also provides a nucleic acid molecule encoding the L-fucose isomerase.

As used herein, the term “nucleic acid molecule” refers to any single or double helix nucleic acid molecule of cDNA, genomic DNA, synthetic DNA or RNA, PNAS or LNA origin, or a mixture thereof. “Nucleic acid” and “polynucleotide” may be used interchangeably in the present application. Since the genetic code is degenerate, one or more codons may be used in order to encode a specific amino acid, and the present invention encompasses polynucleotides encoding a specific amino acid sequence. The aforementioned “nucleic acid molecule” includes a) a nucleic acid base sequence encoding the L-fucose isomerase according to the present invention and a functional equivalent thereof, or b) sequences that hybridize with the sequence under very high stringent conditions. The very high stringent conditions are as described in publicly known literature (Molecular Cloning, Cold Spring Harbor, New York, Cold Spring Harbor Laboratory Press, 1989). Further, the nucleic acid molecule may include a base sequence having a sequence homology of 70% or more, more preferably 80% or more, even more preferably 90% or more and most preferably 95% or more with the nucleic acid base sequences in a) and b). In addition, the nucleic acid molecule may include a fragment or complement of any nucleic acid molecule of a) and b).

The nucleic acid molecule encoding the L-fucose isomerase of the present invention may have a base sequence encoding an L-fucose isomerase represented by the amino acid of SEQ ID NO: 1 and a base sequence having homology with the aforementioned base sequence, and more preferably, may have the base sequence of SEQ ID NO: 2 or a base sequence having homology with the aforementioned base sequence.

The nucleic acid molecule encoding the L-fucose isomerase of the present invention may be obtained from the sequence information disclosed in the present invention by a method for constructing a nucleic acid molecule publicly known in the art, For example, the nucleic acid molecule as a starting material may be isolated from the Raoultella sp. KDH14 strain and/or may be prepared by synthesis based on the base sequence disclosed in the present invention.

The nucleic acid molecule encoding the L-fucose isomerase may be an isolated polynucleotide, that is, a polynucleotide which is substantially free of other chromosomal DNA and RNA, and other base sequences such as extrachromosomal DNA and RNA, but is not limited thereto. A nucleic acid molecule purification method publicly known to those skilled in the art may be used to obtain the isolated nucleic acid molecule. Alternatively, the nucleic acid molecule of the present invention is a functional polynucleotide to which a promoter, a ribosome-binding site and a terminator are operably linked as necessary, in the case of bacterial cells.

The present invention also provides a recombinant vector including the nucleic acid molecule.

As used herein, the term “plasmid”, “vector” or “expression vector” is used interchangeably in the present invention, and refers to a construct for in vivo or in vitro expression. These constructs may be used to insert an L-fucose isomerase-encoding nucleic acid molecule into a host cell. In these constructs, the L-fucose isomerase-encoding nucleic acid is operably linked to an appropriate regulatory sequence so as to be expressed in the host cell, and when inserted into host cells, a vector may replicate and function independently of a host genome, or may be, in some cases, integrated into the host genome itself. Typically, a plasmid vector has a structure including a replication origin that efficiently replicates so as to include several hundreds of plasmid vectors per host cell, an antibiotic resistance gene which allows a host cell inserted into a plasmid to be selected, and a restriction enzyme cleavage site which allows an exogenous nucleic acid molecule to be inserted. Even when there is no appropriate restriction enzyme cleavage site, the vector may be easily ligated with the exogenous nucleic acid molecule using a synthetic oligonucleotide adaptor or a linker according to a typical method.

The “operably linked” means being linked in a manner that enables gene expression when an appropriate nucleic acid molecule binds to a regulatory sequence.

The “recombinant” refers to a cell which replicates a heterologous nucleic acid molecule, or expresses the nucleic acid molecule or expresses a protein encoded by a peptide, a heterologous peptide or a heterologous nucleic acid molecule. Recombinant cells may express genes or gene segments that are not found in the wild-type form of the cells in either the sense or antisense form. Further, recombinant cells may express genes found in cells in the wild-type state, but the genes are modified and reintroduced into cells by artificial means. The “vector” delivers a nucleic acid molecule into cells. The “vector” may further include any operator sequence for regulating transcription, a sequence encoding an appropriate mRNA ribosome binding site and a sequence that regulates the termination of transcription and translation.

Vectors that may be used for expression in host cells are publicly known in the art, and in particular, suitable vectors that may be used for expression in E. coli are also publicly known in the art. According to specific exemplary embodiments of the present invention, pET28a was used as a vector, but the vector is not limited thereto.

The present invention also provides a host cell transformed with the recombinant vector.

As used herein. the term “host cell” includes any cell including the above-described nucleic acid molecule or vector, and may be used for recombinant production of the L-fucose isomerase. As the host cell, a host cell having a high efficiency of introducing a nucleic acid molecule and a high expression efficiency of the introduced nucleic acid molecule is typically used. The host cell includes prokaryotic or eukaryotic cells, and as a recombinant microorganism including these host cells, for example, bacteria, enzymes, molds, and the like may be used, and in the exemplary embodiments of the present invention, E. coli was used, but the host cell is not limited thereto, and any type of microorganism may be used as long as the L-fucose isomerase mutant according to the present invention can be sufficiently expressed.

In the present invention, typically known manipulation methods may be used to insert a nucleic acid molecule into host cells. For example, it is possible to use microprojectile bombardment, particle gun bombardment, silicon carbide whiskers, sonication, electroporation, PEG-mediated fusion, microinjection, a liposome-mediated method, in planta transformation, a vacuum infiltration method, a floral meristem dipping method, and an Agrobacteria spraying method.

The present invention also provides a method for preparing an L-fucose isomerase, the method including: expressing an L-fucose isomerase by culturing the host cell; and obtaining the expressed L-fucose isomerase.

Various culture methods may be applied for (mass) culture of recombinant host cells, for example, large-scale production of gene products expressed or overexpressed from recombinant microorganisms may be achieved by a batch or continuous culture method. Batch and fed-batch culture methods are conventional and known in the art. Methods for regulating nutrients and growth factors for continuous culture processes, as well as techniques for maximizing the formation rate of products are known in the microbial industrial field. In addition, as a culture medium, a medium including a carbon source, a nitrogen source, vitamins and minerals may be used, and a composition may be constituted according to a method known in the art.

The present invention also provides a composition for producing L-fucose including the L-fucose isomerase; and one or more substrates selected from the group consisting of L-fuculose and D-ribulose.

The composition may be a liquid or solid. The composition may also include an L-fucose isomerase alone, may also include other proteins or enzymes together, and may also include an L-fucose isomerase, other proteins, or an additional additive that supplements the stability and/or activity of the enzyme. Examples thereof include glycerol, sorbitol, propylene glycol, salts, sugar, a pH-buffer, a preservative and carbohydrates, but are not limited thereto. Typically, the liquid composition is a water or oil-based slurry, suspension or solvent. The solid composition may be prepared from a liquid composition by spray-drying, lyophilization, down-draught evaporation, thin-layer evaporation, centrifugal evaporation, conveyor drying, or a combination thereof. The solid composition may be granulated to a size suitable for application to food or feed.

Furthermore, the present invention provides a method for producing L-fucose, the method including: reacting the L-fucose isomerase with one or more substrates selected from the group consisting of L-fuculose and D-ribulose.

The reaction may be performed in a buffer solution, and an optimum pH of the L-fucose isomerase may vary depending on the type of buffer solution, but may be about pH 6 to 11. The reaction temperature may be 30 to 50° C. As the enzyme activity drops sharply at 50° C. or more, the L-fucose isomerase is capable of sufficient enzymatic reaction even at room temperature, so there is an advantage in that a process can be economically performed without consuming energy to increase the temperature. Further, the enzyme may be inactivated by heat treatment at a temperature relatively lower than that of existing enzymes.

Therefore, preferably, the reaction may be performed under conditions of 30 to 50° C. and a pH of 6 to 11 as the reaction conditions for 5 minutes to 1 hour.

The buffer solution may be a sodium acetate buffer, a sodium phosphate buffer, a glycine-NaOH buffer, or the like. As an exception, in the case of a Tris-HCl buffer, Tris may act as an inhibitor of the enzyme, so the use thereof needs to be limited.

In addition, as a result of confirming the effects of metal ions on the enzyme activity of the L-fucose isomerase, Mn²⁺, Mg²⁺, Co²⁺, Cd²⁺ and Zn²⁺ increase the enzyme activity, but Fe²⁺, Ca²⁺ and Cu²⁺ inhibit the enzyme activity. Therefore, the production of L-fucose may be enhanced by performing the reaction under a metal ion that increases the enzyme activity.

Hereinafter, the present invention will be described in more detail through the Examples according to the present invention, but the scope of the present invention is not limited by the Examples suggested below.

### EXAMPLE 1

### Microbial Isolation Identification and Gene Identification

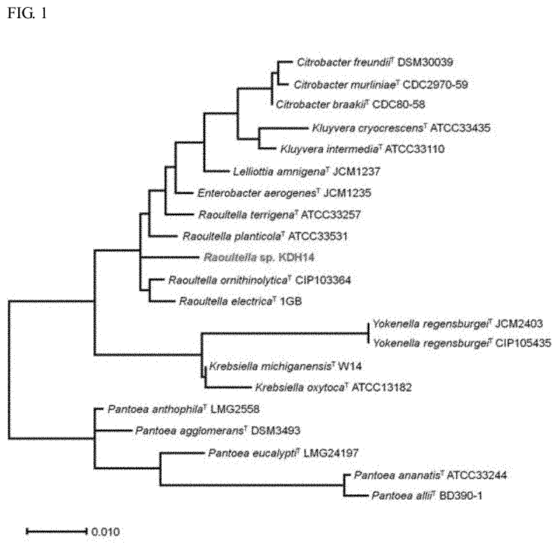

A microbial source of the FucI enzyme of the present invention is a bacterium belonging to the genus Raoultella, and was isolated from abalone intestines based on its ability to grow on fucoidan, which is a constituent of seaweed, as the only carbon source. The isolated strain was identified as a novel species of the genus Raoultella by a 16s rRNA sequencing (the 16S rRNA sequence was amplified by PCR using a bacterial 16s rRNA primer 27F (5′-TTGATCCTGGCTCAG-3′: SEQ ID NO: 3) and 14928 (5′-GGCTACCTTGTTACGACTT-3′: SEQ ID NO: 4), and similarities to RNA sequences in the NCBI database was compared)-based phylogenetic tree, and named Raoultella sp. KDH 14 in the present invention. To find an isomerase of fucose, a genome sequence analysis was performed, and a fucose isomerase was identified by identifying BLAST-based sequence similarity. A strain identification related phylogenetic tree is shown in FIG. 1, and the detailed process of the above contents is as follows.

1) Abalone intestines were decomposed, diluted with water, and inoculated into a M9 minimal medium containing 2% fucoidan.

2) The inoculated cells were cultured at 30° C.

3) Cell growth was confirmed by a Synergy HTX multi-mode reader, and a strain with a high growth rate was isolated.

### EXAMPLE 2

### Cloning, Expression and Purification of Rd FucI

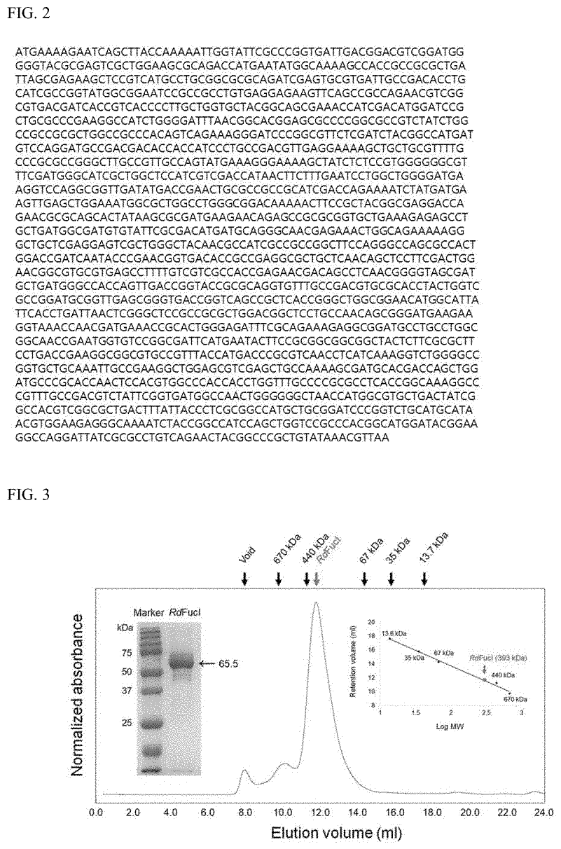

In the cloning of the RdFucI gene used in the present invention, genomic DNA was extracted from the isolated Raoultella sp. KDH 14, and then a target gene was amplified by a polymerase chain reaction. Cloning was completed by inserting the amplified target gene into an expression vector pET28a. FIG. 2 shows a base sequence of the gene.

E. coli BL21 (DE3) was used as a recombinant enzyme protein expression host. After E. coli cells were transformed with the recombinant gene, the E. coli cells were seed-cultured in 20 mL of a liquid medium (LB broth) for about 16 hours. The E. coli cells were re-inoculated into 1 L of LB, further cultured at 37° C., and when the value of OD600 reached about 0.6 to 0.8, the temperature was lowered to 16° C., and expression was induced by adding isopropyl β-d-1-thiogalactopyranoside (IPTG) thereto, such that the final concentration was 1 mM. After IPTG was added, Escherichia coli was further cultured for about 16 hours, and then the cells were recovered by centrifugation. 50 μg/mL of kanamycin was used as a selection marker throughout the process. The recovered cells were disrupted by an ultrasonicator, and then a crude protein in the cells was extracted and purified. The purification process was performed by affinity chromatography using a His-trap column, and most of the target protein was recovered from 300 mM imidazole.

As illustrated in FIG. 3, an enzyme protein with a size of 65.5 kDa was confirmed, and was composed of 591 amino acids.

### EXAMPLE 3

### Reversible Reaction of Enzyme

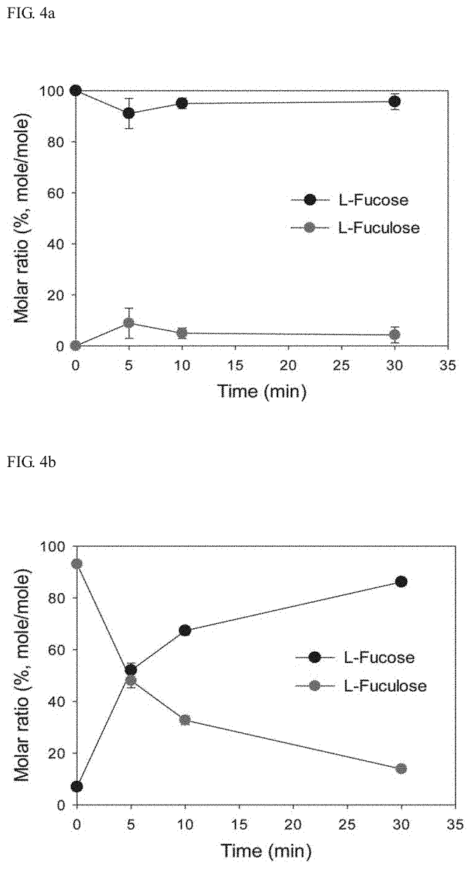

In order to identify what kind of reaction RdFucI favored in a reaction between fucose and fuculose, a reversible reaction of the recombinant enzyme was investigated over time. For this purpose, 10 mM L-fucose (FIG. 4a) or L-fuculose (FIG. 4b) included in 20 mM sodium phosphate (pH7) was used as a substrate, and was each reacted with 1.5 μg of RdFucI at 30° C. in the presence of 1 mM MnCl₂, and L-fucose was experimentally quantified. In contrast, L-fuculose was expressed as a calculated value.

As a result, the reaction rate of the reverse reaction was about 5-fold faster, and the ratio of fucose and fuculose in the equilibrium state was about 90:10 regardless of whether fucose or fuculose was used as a substrate. This means that the catalytic reaction by RdFucI favors the reverse reaction, that is, a direction of producing fucose.

### EXAMPLE 3

### Investigation of Biochemical Properties (Temperature, pH, and Metal Ion) of Rd FucI for Fuculose

For the potential applicability of RdFucI, various biochemical properties needed to be investigated using fuculose as a substrate.

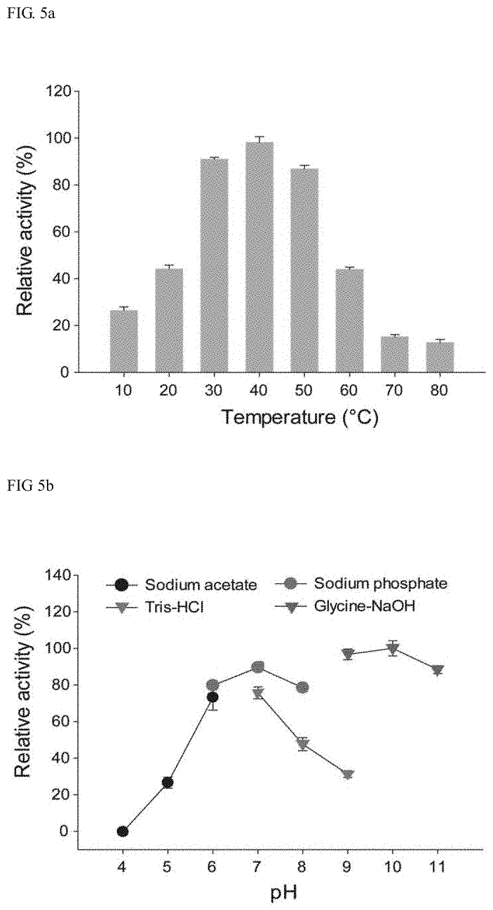

1) Temperature effect: As a result of treating 10 mM of a substrate with 1.5 μg of an enzyme at a pH of 7 at various temperatures (10 to 80° C.), the highest activity was exhibited at 40° C., and the maximum approximations were exhibited at 30, 40, and 50° C. (80% or more of the maximum activity) (FIG. 5a).

2) pH effect: As a result of treating 10 mM of a substrate with 1.5 μg of an enzyme under various pH conditions (pH 4 to 11:50 mM sodium acetate (pH 4 to 6), 50 mM sodium phosphate (pH 6 to 8), 50 mM Tris-HCl (pH 7 to 9), 50 mM glycine-NaOH (pH 9 to 11)) at 40° C., the highest activity was exhibited under an alkaline condition (pH 9 and 10), and the maximum approximations were also exhibited at a pH of 6, 7, 8, and 11. Meanwhile, when a Tris-HCl buffer was used at a pH of 7, 8, and 9, the activity was much lower than that when other buffer types were used at the same pH, and this suggests that Tris may act as an inhibitor of the enzyme, implying that the Tris buffer should be avoided when the present enzyme is studied or applied (FIG. 5b).

3) Metal ion effect: Sugar isomerases generally require a metal ion as a cofactor. Therefore, the effects of various metal ions on the activity of RdFucI were also studied in the present invention (Table I). As a result, the activity of the enzyme was increased about 7-fold higher in the presence of Mn²⁺.

TABLE 1
Effect of metal ion

| Metal ion | Relative activity (%) |
|---|---|
| Control | 100 ± 3 |
| EDTA | 77 ± 12 |
| MnCl₂ | 738 ± 70 |
| MgCl₂ | 533 ± 31 |
| CoCl₂ | 353 ± 83 |
| CdCl₂ | 183 ± 9 |
| ZnCl₂ | 159 ± 4 |
| CsCl₂ | 101 ± 16 |
| LiSO₄ | 101 ± 15 |
| NiCl₂ | 91 ± 13 |
| FeCl₃ | 74 ± 3 |
| CaCl₂ | 64 ± 8 |
| CuCl₂ | 58 ± 7 |

### EXAMPLE 4

### Substrate Specificity of Rd FucI

In general, sugar isomerases show reactivity for various sugars. As a result of testing with various aldoses (L-fucose, D-arabinose, D-altrose, D-galactose, D-mannose and D-glucose) and ketose substrates (L-fuculose, D-ribulose, D-psicose, D-tagatose and D-fructose), the highest activity was shown against L-fuculose (115.3 U/mg) and D-ribulose (127.3 U/mg), showing much higher activities compared to activities for the other tested aldose and ketose substrates. Furthermore, only in the case of L-fuculose and D-ribulose, RdFucI showed a much more dominant reaction on the ketose substrate than on the aldose substrate (FIG. 6).

### EXAMPLE 5

### Crystallization of Rd FucI and Data Collection

Crystal screening was performed by a sitting-drop vapor diffusion method at 20° C. using a commercially available kit Index HT, Salt RX HT, and Crystal Screen HT. Microcrystals were primarily produced in a solution containing 0.1 M HEPES (pH7.5), 20% (w/v) polyethylene glycol 10,000. High-quality crystals were obtained from the same solution by a hanging drop method. The crystals were immersed in a reservoir solution containing an additional 20% (v/v) glycerol and flash-cooled in a nitrogen stream. X-ray diffraction datasets for crystals were collected at 100 K from beamline 11C of PLS-II using Pilatus 6M or from beamline 6A using an ADSC Quantum Q270 CCD detector. Diffraction data was processed using the HKL2000 program.

### EXAMPLE 6

### Overall Structure of Rd FucI

The phase was solved using a molecular replacement method implemented in MOLREP using the crystal structure of EcFucI (PDB code: 1FUI) as a search model. The structure was manually reconstructed and purified using COOT. Structural purification was performed using the phenix.refine program PHENIX. Structural quality was verified using MolProbity.

Monomeric RdFucI includes 19 α-helices and 23 β-strands, including N1, N2 and C domains (FIG. 7a). The N1 domain (Ser5-Met172) adopts an α/β-fold and is involved in substrate recognition of the hexamer formation of RdFucI. The N2 domain (Lys173-Leu352) and the C domain (Thr353-Arg591) include metal binding residues involved in catalytic activity (FIG. 7a). The RdFucI subunit forms D3h virtual symmetry with a dimer of a trimer.

### EXAMPLE 7

### Structure-Based Investigation of Active Site and Binding Position of Rd FucI

The substrate binding pocket is formed by the N2 and C domains of Subunit A and the N1 domain of Subunit B (FIGS. 8a to 8c), and has a total of 6 substrate binding sites in homohexameric RdFucI. An entrance of a substrate binding pocket to which a substrate approaches is about 11×12.5 Å (FIG. 8a). A substrate binding pocket, in which a metal binding site is formed, has a negatively-charged surface of about 4×5 Å (FIG. 8b). The distance between the metal binding site and the substrate binding pocket is about ˜16.7 Å (FIG. 8d), meaning that the active center is located deep in the pocket. This shows that both the open shape and ring shape of the substrate can access the active site center, but this also shows that bulk saccharides cannot access an active site present inside the substrate binding pocket.

### EXAMPLE 8

### Identification of Specificity of Rd FucI through Structural Comparison

Using the DALI server, RdFucI is L-FucIs (EcFucI, PDB code 1FUI, Z score: 60.6, in the case of 587Cαs, rmsd: 0.3) of Escherichia coli, Aeribacillus pallidus (ApFucI, 3A9R, Z score: 56.6, in the case of 580CαS, rmsd: 0.7), Streptococcus pneumonia (SpFucI, 4020, Z score: 55.9, rmsd: in the case of 585 Cαs, 0.7). The superimposition of the substrate binding pockets shows that substrate recognition residues (Arg16, W88, Gln300, Tyr437, Trp496 and Asn524) exhibit minor three-dimensional structures, whereas metal binding residues Glu337, Asp361, and His528 (numbered in RdFucI) are positionally identical to other proteins. In contrast, the α7-α8 loop of each L-FucI placed on the surface of the substrate binding pocket has a different conformation. Although the sequence alignment of L-FucI exhibits high similarity, the sequence of the α7-α8 loop of each L-FucI is very diverse (FIG. 9b). Each L-FucI forms a unique substrate binding pocket because the α7-α8 loop is involved in the structure formation of the substrate binding pocket (FIG. 9c). Although L-FucI generally has a negatively charged surface around the metal binding site, the surface of the substrate binding pocket exhibits a different charge state (FIG. 9c). As a result, a difference in the α7-α8 loop structure will cause a difference in the substrate specificity of L-FucI.

The L-fucose isomerase of the present invention can be applied to the field of L-fucose production.

What is claimed is:

1. An L-fucose isomerase which comprises the amino acid sequence of SEQ ID NO: 1 and favors a conversion reaction from L-fuculose to L-fucose in a reversible reaction between L-fucose and L-fuculose.

2. The L-fucose isomerase of claim wherein the L-fucose isomerase is derived from a Raoultella sp. KDH 14 strain.

3. The L-fucose isomerase of claim 1, wherein activity of the L-fucose isomerase is increased by one or more metal ions selected from the group consisting of Mn²⁺, Mg²⁺, Co²⁺, Cd²⁺ and Zn²⁺.

4. The L-fucose isomerase of claim 1, wherein activity of the L-fucose isomerase is inhibited by one or more metal ions selected from the group consisting of Fe²⁺, Ca²⁺ and Cu²⁺.

5. A nucleic acid molecule encoding the L-fucose isomerase of claim 1.

6. The nucleic acid molecule of claim 5, wherein the nucleic acid molecule comprises the base sequence of SEQ ID NO: 2.

7. A recombinant vector comprising the nucleic acid molecule of claim 6.

8. A host cell transformed with the recombinant vector of claim 7.

9. A method for preparing an L-fucose isomerase, the method comprising: expressing an L-fucose isomerase by culturing the host cell of claim 8; and obtaining the expressed L-fucose isomerase.

10. A composition for producing L-fucose comprising the fucose isomerase of claim 1; and one or more substrates selected from the group consisting of L-fuculose and D-ribulose.

11. The composition of claim 10, further comprising one or more metal ions selected from the group consisting of Mn²⁺, Mg²⁺, Co²⁺, Cd²⁺ and Zn²⁺.

12. A method for producing L-fucose, the method comprising: reacting the L-fucose isomerase of claim 1 with one or more substrates selected from the group consisting of L-fuculose and D-ribulose.

13. The method of claim 12, wherein the reaction is performed under conditions of 30 to 50° C. and a pH of 6 to 11 for 5 minutes to 1 hour.

14. The method of claim 12, wherein the reaction is performed under one or more metal ions selected from the group consisting of Mn²⁺, Mg²⁺, Co²⁺, Cd²⁺ and Zn²⁺.


SEQUENCE LISTING (4 sequences)

SEQ ID NO: 1 - protein, 591 aa, Raoultella sp. KDH 14
MKRISLPKIGIRPVIDGRRMGVRESLEAQTMNMAKATAALISEKLRHACGAQIECVIADTCIAGMAESAACEEKFSRQNVGVTITVTPCWCYGSETIDMDPLRPKAIWGFNGTERPGAVYLAAALAAHSQKGIPAFSIYGHDVQDADDTTIPADVEEKLLRFARAGLAVASMKGKSYLSVGGVSMGIAGSIVDHNFFESWLGMKVQAVDMTELRRRIDQKIYDEVELEMALAWADKNFRYGEDQNAQHYKRDEEQSRAVLKESLLMAMCIRDMMQGNEKLAEKGLLEESLGYNAIAAGFQGQRHWTDQYPNGDTAEALLNSSFDWNGVREPFVVATENDSLNGVAMLMGHQLTGTAQVFADVRTYWSPDAVERVTGQPLTGLAEHGIIHLINSGSAALDGSCQQRDEEGKPTMKPHWEISQKEADACLAATEWCPAIHEYFRGGGYSSRFLTEGGVPFTMTRVNLIKGLGPVLQIAEGWSVELPKAMHDQLDARTNSTWPTTWFAPRLTGKGPFADVYSVMANWGANHGVLTIGHVGADFITLAAMLRIPVCMHNVEEGKIYRPSSWSAHGMDTEGQDYRACQNYGPLYKR

SEQ ID NO: 2 - DNA, 1776 bp, Raoultella sp. KDH 14
atgaaaagaatcagcttaccaaaaattggtattcgcccggtgattgacggacgtcggatgggggtacgcgagtcgctggaagcgcagaccatgaatatggcaaaagccaccgccgcgctgattagcgagaagctccgtcatgcctgcggcgcgcagatcgagtgcgtgattgccgacacctgcatcgccggtatggcggaatccgccgcctgtgaggagaagttcagccgccagaacgtcggcgtgacgatcaccgtcaccccttgctggtgctacggcagcgaaaccatcgacatggatccgctgcgcccgaaggccatctggggatttaacggcacggagcgccccggcgccgtctatctggccgccgcgctggccgcccacagtcagaaagggatcccggcgttctcgatctacggccatgatgtccaggatgccgacgacaccaccatccctgccgacgttgaggaaaagctgctgcgttttgcccgcgccgggcttgccgttgccagtatgaaagggaaaagctatctctccgtggggggcgtttcgatgggcatcgctggctccatcgtcgaccataacttctttgaatcctggctggggatgaaggtccaggcggttgatatgaccgaactgcgccgccgcatcgaccagaaaatctatgatgaagttgagctggaaatggcgctggcctgggcggacaaaaacttccgctacggcgaggaccagaacgcgcagcactataagcgcgatgaagaacagagccgcgcggtgctgaaagagagcctgctgatggcgatgtgtattcgcgacatgatgcagggcaacgagaaactggcagaaaaagggctgctcgaggagtcgctgggctacaacgccatcgccgccggcttccagggccagcgccactggaccgatcaatacccgaacggtgacaccgccgaggcgctgctcaacagctccttcgactggaacggcgtgcgtgagccttttgtcgtcgccaccgagaacgacagcctcaacggggtagcgatgctgatgggccaccagttgaccggtaccgcgcaggtgtttgccgacgtgcgcacctactggtcgccggatgcggttgagcgggtgaccggtcagccgctcaccgggctggcggaacatggcattattcacctgattaactcgggctccgccgcgctggacggctcctgccaacagcgggatgaagaaggtaaaccaacgatgaaaccgcactgggagatttcgcagaaagaggcggatgcctgcctggcggcaaccgaatggtgtccggcgattcatgaatacttccgcggcggcggctactcttcgcgcttcctgaccgaaggcggcgtgccgtttaccatgacccgcgtcaacctcatcaaaggtctggggccggtgctgcaaattgccgaaggctggagcgtcgagctgccaaaagcgatgcacgaccagctggatgcccgcaccaactccacgtggcccaccacctggtttgccccgcgcctcaccggcaaaggcccgtttgccgacgtctattcggtgatggccaactggggggctaaccatggcgtgctgactatcggccacgtcggcgctgactttattaccctcgcggccatgctgcggatcccggtctgcatgcataacgtggaagagggcaaaatctaccggccatccagctggtccgcccacggcatggatacggaaggccaggattatcgcgcctgtcagaactacggcccgctgtataaacgttaa

SEQ ID NO: 3 - DNA, 20 bp, Artificial Sequence
  16S rRNA primer,  27F
agagtttgatcctggctcag

SEQ ID NO: 4 - DNA, 19 bp, Artificial Sequence
  16S rRNA primer, 1492R
ggctaccttgttacgactt